(19) United States
(12) Patent Application Publication
(10) Pub. No.: US 2023/0402134 A1
(43) Pub. Date: Dec. 14, 2023

(54) SYSTEM FOR PREDICTING SENSORY ATTRIBUTES OR PHYSICO-CHEMICAL PROPERTIES OF AN OIL OR A MIXTURE OF OILS FOR PERSONAL CARE

(71) Applicant: BASF SE, Ludwigshafen (DE)

(72) Inventors: Sandip Bhattacharya, Düsseldorf-Holthausen (DE); Matthias Hloucha, Düsseldorf-Holthausen (DE); Daniela Prinz, Dormagen (DE); Wolf Eisfeld, Düsseldorf-Holthausen (DE)

(21) Appl. No.: 18/032,219

(22) PCT Filed: Oct. 22, 2021
(86) PCT No.: PCT/EP2021/079323

§ 371 Date: Apr. 17, 2023

(30) Foreign Application Priority Data
Oct. 22, 2020 (EP) 20203303.1

(51) Int. Cl. G16C 20/30; G16C 20/70; G16C 60/00

(52) U.S. Cl. CPC G16C 20/30; G16C 20/70; G16C 60/00

(57) ABSTRACT

A system (110) for predicting sensory attributes or physico-chemical properties of an oil or a mixture of oils for personal care is proposed. The system (110) comprises at least one communication interface (112) for providing data and at least one processing device (114). The processing device (114) is configured for: —obtaining via the communication interface (112) of at least one physico-chemical property of the oil or the mixture of oils or of at least one sensory attribute and of at least one model relating one or more physico-chemical properties to one or more sensory attributes; —determining at least one sensory attribute of the oil or the mixture of oils based on the obtained physico-chemical properties and the model or determining at least one physico-chemical property of the oil or the mixture of oils based on the obtained sensory attributes and the model; —providing via the communication interface (112) the determined sensory attribute or the determined physico-chemical property.

### TECHNICAL FIELD

The invention relates to a system, a computer-implemented method and a computer program product for predicting sensory attributes or physico-chemical properties of an oil or a mixture of oils for personal care, more particular for cosmetics. Other applications are possible.

### BACKGROUND ART

Products in cosmetic industries widely contain mixtures of multiple ingredients including oils. In personal care industry a frequent term for oils is emollients. Oils play an essential part for the moistening effect and are also influencing the resorption of the cosmetic.

A clear view of requirements of consumers and identifying of future or new trends are critical for development of successful personal care products. With customized products and solutions for cosmetics, opportunities and possibilities lying within these future or new trends should be exhausted.

Future or new trends also include the development of new formulations for oil-containing products for cosmetics. Changes in formulations may have a tremendous influence on wearing and application comfort of cosmetic products and impart an impression to the consumer regarding sensory properties of the cosmetic product.

New formulations for oil-containing products for cosmetics are tested by time consuming and expensive tests. Specifically, sensory tests for example require trained human testers, that apply the new formulation to the skin and then rate on a scale the sensory values for a formulation. Sensory results therefore also have statistical errors due to human evaluation of attributes. Extensive tests on physico-chemical properties on the new formulation for oil-based products are also required when developing a new formulation. Physico-chemical properties however are evaluated by well established methods and have comparatively little errors in their measurement.

European Patent application No. EP 20 163 091.0 and WO2021/180922 describe a computer implemented method for determining performance properties of an oil-containing product for cosmetics based on a data driven model and/or the rigorous model and composition parameters.

Thus, there is a need for systems and methods for predicting sensory attributes or physico-chemical properties of an oil or a mixture of oils for personal care.

Nakano K. et al. “A neural network approach to predict tactile comfort of applying cosmetic foundation”, TRIBOLOGY INTERNATIONAL, ELSEVIER LTD, AMSTERDAM, NL, vol. 43, no.11, 2010 Nov. 1, pages 1978-1990, XP027243450, ISSN: 0301-679X describes an expert system developed to predict the degree of tactile comfort during the application of cosmetic foundation. Gilbert L. et al. “Predicting sensory texture properties of cosmetic emulsions by physical measurements”, CHEMOMETRICS AND INTELLIGENT LABORATORY SYSTEMS, vol. 124, 1 May 2013, pages 21-31, XP055069346, ISSN: 0169-7439, DOI: 10.1016/j.chemolab.2013.03.002 describes instrumental measurements for predicting the sensory texture of cosmetic emulsions. R. Kora et al. “Sensory and instrumental characterization of fast inverting oil-in-water emulsions for cosmetic application”, INTERNATIONAL JOURNAL OF COSMETIC SCIENCE, vol. 38, no. 3, 1 Jun. 2016, pages 246-256, XP055419978, NL ISSN: 0142-5463, DOI: 10.1111/ics. 12282 describes a study performing short-term sensory testing and instrumental (conductivity and rheological) characterization of a fast inverted oil-in-water emulsion base.

### Problem to be Solved

It is therefore desirable to provide methods and devices which address the above-mentioned technical challenges. Specifically, devices and methods shall be provided which allow for reliably predicting sensory attributes.

### SUMMARY

This problem is addressed by a method and a device for predicting sensory attributes or physicochemical properties of an oil or a mixture of oils for personal care with the features of the independent claims. Advantageous embodiments which might be realized in an isolated fashion or in any arbitrary combinations are listed in the dependent claims as well as throughout the specification.

In a first aspect of the present invention, a system for predicting sensory attributes or physicochemical properties of an oil or a mixture of oils for personal care is proposed.

Oils in the sense of that application may comprise cosmetic oil components. Cosmetic oil components may be oil components selected from the group consisting of fatty acid esters, esters of C6-C28 fatty acids and C6-C28 fatty alcohols, glyceryl esters, fatty acid ester ethoxylates, alkyl ethoxylates, C12-C28 fatty alcohols, C12-C28 fatty acids, Guerbet esters, Guerbet alcohols and Guerbet acids, saturated alkanes, C12-C28 fatty alcohol ethers, vegetable oils, natural essential oils, mineral oil, parafinum liquidum, petrolatum, isoparaffins, preferably from the group consisting of dibutyl adipate (Cetiol® B), phenethyl benzoate, coco-caprylate (Cetiol® C5), coco-caprylate/caprate (Cetiol® LC, Cetiol® C5, Cetiol® C 5C), propylheptyl caprylate (Cetiol® Sensoft), caprylyl caprylate/caprate (Cetiol® RLF), myristyl myristate (Cetiol® MM), capric glycerides, coco-glycerides (Myritol® 331), capryl/caprin-triglyceride (Myritol® 312), capryl/caprin-triglyceride (Myritol® 318), C12-15 alkyl benzoate (Cetiol® AB), PPG-3 benzyl ether myristate, C12-13 alkyl lactate, isodecyl salicylate, alkyl malate, isoamyl laurate, propylheptyl caprylate, butyloctyl salicylate, polycrylene, dicaprylyl carbonate (Cetiol® CC), dicaprylyl ether (Cetiol® OE), 2-octyldodecylmyristate, isohexadecane, dimethyl capramide, squalene, isopropyl isostearate, isostearyl isostearate, decyl oleate (Cetiol® V), oleyl erucate (Cetiol® J 600), cetearyl ethylhexanoate (Luvitol® EHO), octyldodecanol (Eutanol® G), hexyldecanol (Eutanol® G16), volatile linear C8 to C16 alkanes, C10 to C15 alkanes, C11-C13 alkanes (Cetiol® Ultimate), C13-15 alkanes, C15-19 alkanes, C17-23 alkanes, isododecane, undecane, tridecane (Cetiol® Ultimate), dodecane, propylene glycol dipelargonate, diisopropyl sebacate, cetearyl isononanoate (Cetiol® SN), isononyl isononanoate, isocetyl stearoyl stearate, dipentaerithrityl hexacaprylate/hexacaprate, isodecyl neopentanoate, PEG-6 caprylic/ capric glycerides (Cetiol® 767), caprylic/capric triglyceride (Myritol® 312, Myritol® 318), ethylhexyl stearate, ethylhexylcocoate, ethylhexyl stearate (Cetiol® 868), dipropylheptyl carbonate (Cetiol® 4 All), hexyl laurate (Cetiol® A), dicaprylyl carbonate, PEG-7 glyceryl cocoate (Cetiol® HE), polyglyceryl-3 diisostearate (Lameform® TGI), lauryl alcohol, methyl canolate (Cetiol® MC), hexyldecyllaurate and hexyldecanol (Cetiol® PGL), hexyldecyl stearate (Eutanol® G 16S), PPG-15 Stearylether (CETIOL® E), ethylhexyl palmitat (CEGESOFT® C24). Further fatty acid esters may be myristyl palmitate, myristyl stearate, myristyl isostearate, myristyl oleate, myristyl behenate, myristyl erucate, myristyl myristate (Cetiol® MM), cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl oleate, isostearyl behenate, isostearyl oleate, oleyl myristate, oleyl palmitate, oleyl stearate, oleyl isostearate, oleyl oleate, oleyl behenate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are Guerbet alcohols, Guerbet acids, Guerbet esters, preferably Guerbet esters of linear C6-22 fatty acids with branched alcohols, especially Guerbet esters of linear C6-22 fatty acids with branched alcohols with C6 -C18, preferably C8 -C10 fatty alcohols more particularly 2-ethyl hexanol, esters of C18-38 alkylhydroxy carboxylic acids with linear or branched C6-22 fatty alcohols, more especially Dioctyl Malate, esters of linear and/or branched fatty acids with polyhydric alcohols (for example propylene glycol, dimer diol or trimer triol) and/or Guerbet alcohols, triglycerides based on C6-10 fatty acids, liquid mono-, di-and triglyceride mixtures based on C6-18 fatty acids, esters of C6-22 fatty alcohols and/or Guerbet alcohols with aromatic carboxylic acids, more particularly benzoic acid, esters of C2-12 dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, Guerbet carbonates based on fatty alcohols containing 6 to 18 and preferably 8 to 10 carbon atoms, esters of benzoic acid with linear and/or branched C6-22 alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols.

Further suitable oil components may be natural products selected from the group of Elaeis guiineensis oil (Cegesoft® GPO), Passiflora incarnata seed oil (Cegesoft® PFO), olive oil, olus oil (Cegesoft® PS6), Butyrospermum parkii butter (Cetiol® SB 45), ethylhexylcocoat (and) Cocos Nucifera 01 (CETIOL® COCO), Shorea stenoptera seed butter (Cegesoft® SH), almond oil, avocado oil, borage oil, canola oil, castor oil, chamomile, coconut oil, corn oil, cottonseed oil, jojoba oil, evening primrose oil, papaya oil, palm oil, hazelnut oil, peanut oil, walnut oil, safflower oil, sesame oil, soybean oil, sunflower oil, sweet almond, a rice bran/wheat germ oil, rosehip oil, Ricinus communis oil, lanolin; hydrogenated vegetable oil, Candelilla cera, Euphorbia vegetable oil (Cegesoft® VP), sterols and derivatives.

In addition, silicones and silicone derivatives such as polydimethylsiloxanes, methicone, dimethicone, cyclomethicone, caprylyl methicone, dimethicone copolyol, undecylcrylene dimethicone, dimethiconol, trimethicone, organo-siloxanes may be used as oil components in cosmetic compositions.

The term “mixture” of oils as used herein is a broad term and is to be given its ordinary and customary meaning to a person of ordinary skill in the art and is not to be limited to a special or customized meaning. The term specifically may refer, without limitation, to a combination of two or more different oils. The mixture of oils may comprise at least two different oils, at least three different oils or at least four different oils. Specifically, the personal care product may comprise at least two different oils. Specifically, the personal care product may comprise at least three different oils. Even other mixtures are possible. By combining two, three or even more oils to create a new personal care product a large variation of sensor attributes for the new personal care product can be achieved.

The different oils may be present in the mixture with a certain ratio. The ratio may be a relationship in quantity, amount, or size between two or more of the different oils. The ratio may be the relative or absolute. The ratio of the different oils may be e.g. weight percentage, volume percentage, mixing ratio, molar ratio.

The term “system” as used herein is a broad term and is to be given its ordinary and customary meaning to a person of ordinary skill in the art and is not to be limited to a special or customized meaning. The term specifically may refer, without limitation, to an arbitrary set of interacting components or parts forming a whole or entity. Specifically, the components may interact with each other in order to fulfil at least one common function. The components may be handled independently or may be coupled or connectable to one another. The system may be a recommendation system and/or a validation system. The term “recommendation system” as used herein is a broad term and is to be given its ordinary and customary meaning to a person of ordinary skill in the art and is not to be limited to a special or customized meaning. The term specifically may refer, without limitation, to a system designed for providing at least one recommendation, such as to at least one user. For example, the recommendation may be a formula e.g. ingredients of a mixture of oils. The term “validation system” as used herein is a broad term and is to be given its ordinary and customary meaning to a person of ordinary skill in the art and is not to be limited to a special or customized meaning. The term specifically may refer, without limitation, to a system designed for determining whether a target, such as at least one target sensory attribute is reached at least within predefined tolerances.

The term “personal care” as used herein is a broad term and is to be given its ordinary and customary meaning to a person of ordinary skill in the art and is not to be limited to a special or customized meaning. The term specifically may refer, without limitation, to consumer products used in one or more of personal hygiene, cosmetic such as for hair care or body care, in particular skin care, and the like. Cosmetics are personal care products. Cosmetics may be used for different field of uses, in particular are applicable to different parts of the human body. Specifically, they may include, but are not limited to, products that can be applied to the face, to the body, to the hands and/or nails, to the feet, to the hair and to the mouth. Examples for cosmetics are skin care creams, sun screen, lipsticks, deodorants, lotions, powders, perfumes, baby products, bath oils, bubble baths, fingernail and toe nail polish, and hand sanitizer; hair dye, hair sprays, gels shampoo conditioner, bath salts, and body butters. Cosmetics often contain oil or a mixture of different oils. Generally, for cosmetics, the user experience is highly important.

A measure for user experience may be sensory attributes. The term “sensory attribute” as used herein is a broad term and is to be given its ordinary and customary meaning to a person of ordinary skill in the art and is not to be limited to a special or customized meaning. The term specifically may refer, without limitation, to features characterizing perception of the oil or the mixture by human senses when used in the respective field of use. In principle, any sensory attribute can be defined as the amount of “sensory attribute” perceived on the skin. Prerequisite may be that the panelists are thoroughly trained on the particular sensory attribute. In particular, sensory attributes may determine how a product for personal care comprising the oil or oil mixture, in particular cosmetics comprising the oil or oil mixture, is perceived on human skin. Generally a plurality of sensory attributes may be suitable for characterizing perception of the oil or the mixture by human senses when used in the respective field of use. Sensory attributes may comprise at least one attribute selected from the group consisting of: wetness, spreadability, thickness, absorption, distribution, oily, greasy, amount of residue, thickness of residue, slipperiness, smoothness, stickiness, dryness, gloss, rubs to absorbency, silicone feel, powdery feel and the like. These examples may be, in particular, beneficial as they are well suited to describe the user experience of a cosmetic product. However, other sensory attributes are possible. A sensory attribute may refer to data indicative of a sensory attribute.

These properties may be classified into those measured immediately after application, during the rub-out phase and after N minutes after application. Here N can be but is not limited to 5 minutes, 20 minutes, etc.

The sensory attributes may be measured by trained panelists, wherein test conditions with respect to temperature, humidity and the like are selected according to “Basic Principles of Sensory Evaluation”, ASTM Special Technical Publication 433 (1969) and/or DIN 10950:2020-09, “Sensorische Prüfung-Allgemeine Grundlagen incl. Anforderungen an die Räumlichkeiten”.

The individual sensory attributes are self-explanatory in accordance to the respective names. Sensory attributes may be defined differently in different labs. The sensory attributes may be defined as described in “Sensory Evaluation Techniques”, Morten C. Meilgaard, Gail Vance Civille, B. Thomas Carr, 5th edition, CRC Press, Taylor & Francis Group, Boca Raton, ISBN: 9781-4822-1691-2 (e-book) 2016. For example, the sensory attributes may be defined as follows.

The sensory attribute “wetness” may refer to an amount of water which is perceived. An oil can have a wet appearance if it is very thin and is a little cooling.

The sensory attribute “spreadability” may refer to a rub-out phase during distribution. The sensory evaluation is done regarding an easy moving on skin (e.g. like Cetiol® Ultimate) or a difficult spreading (e.g. like Cegesoft® PS6).

The sensory attribute “thickness” of the film may refer to a residue of a high layer or a low layer. If the panelist can feel directly the skin surface or if there is a film between fingertip and skin surface.

The sensory attribute “oily”, or “oil” or oiliness may refer to thin or rich oils. The amount of oil during rub-out is determined. A thin, pliable coating is felt that is slippery and provides a smooth, continuous feel.

The sensory attribute “stickiness” may refer to tackiness of the oil on the skin and may be determined after absorption. The degree to which fingers adhere to residual product may be assessed.

The sensory attribute “amount of residue” may refer to an amount of product that is felt on the skin immediately after absorption.

The sensory attribute “thickness of residue” may be determined after the oil is absorbed, in particular time immediately after absorption, like in the rub-out phase.

The sensory attribute “powdery feel” may refer to a measure for very dry, smooth and silky feel on the skin. There is a thin, slippery coating on the skin that leaves a very dry, smooth and silky feel on the skin. It reminds of talcum powder or baby powder or corn starch.

The sensory attribute “silicone feel” may refer to a pliable coating that provides a continuous feel that fills in the texture of the skin and obscures fingerprint feel. Can be thick or thin. The coating is usually very persistent and hard to remove, if there is any. Reminiscent of silicone oil feel.

The sensory attribute “smoothness” may be determined directly after the oil is absorbed and may refer to a degree to which the skin feels smooth while gliding over the arm is evaluated.

The sensory attribute “dryness”, also denoted as “wax-dry”, may be determined after the oil is absorbed. A thin, stiff coated feel that is draggy, but not slippery. It provides a smooth feel on the skin with an occlusive barrier. It can be also dry with a hint of drag, reminiscent of a wax candle.

The sensory attribute “greasy”, also denoted cushiony, of the skin may be determined after 15-20 rubs. If there is a nourishing feel or a soft feel. The feeling is a reminiscent of a pillow.

The sensory attribute “slipperiness”, also referred to as “gliding”, may refer to a measure of an ease of moving fingers across the skin.

The sensory attribute “rubs to absorbency” may refer to a number of rubs the feeling where until the panelist feels that the oil is absorbed.

The sensory attribute “gloss” may be determined after the oil is absorbed. The panelist may assess if the skin is shiny, e.g. like satin.

As used herein, the term “sensory attributes of the oil or the mixture of oils” may refer to sensory attributes of the oil or of different oils of the mixture of oils.

Usually, sensory attributes have to be measured by trained panelists and are not available from datasheets/databases. The number of panelists may be about 10 to 100 more particular about 20. It turned out that reliable information can already be derived with about 10 trained panelists. For each sensory attribute, the panelists rate the personal care product. This rating may be done on a monadic scale, such as from 0 to 100. The final value of a sensory attribute for rate of the personal care product may be taken as an average or the median value from all panelists. Since sensory attributes are evaluated by humans, albeit trained, its values can have a large scattering, in particular statistical divergence. Moreover, however, currently, the sensory attributes of new products for personal care, in particular cosmetics, have to be determined by tests which are time consuming and costly. The proposed system allows for predicting sensory attributes and, thus, may allow that the number of tests required for a new personal care product, in particular cosmetics, can be greatly reduced or the tests can be even eliminated. The system according to the present invention may allow objectifying the sensor attributes. This may allow comparison of sensory attributes such as a determined sensory attribute to a target sensor attribute. Moreover, it may be possible to waive and/or prevent need of tests using trained panelists, and, thus, may allow significantly reducing costs.

The term “predicting sensory attributes” as used herein is a broad term and is to be given its ordinary and customary meaning to a person of ordinary skill in the art and is not to be limited to a special or customized meaning. The term specifically may refer, without limitation, to a process of determining expected sensory attributes based on at least one model.

The system comprises at least one communication interface for providing data and at least one processing device. The processing device is configured for:
obtaining via the communication interface of at least one physico-chemical property of the oil or the mixture of oils or of at least one sensory attribute and of at least one model relating one or more physico-chemical properties to one or more sensory attributes;
determining at least one sensory attribute of the oil or the mixture of oils based on the obtained physico-chemical properties and the model or determining at least one physicochemical property of the oil or the mixture of oils based on the obtained sensory attributes and the model;
providing via the communication interface the determined sensory attribute or the determined physico-chemical property.

The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. The communication interface, in particular, to transfer information between two units of a computational device such as between a database and a processing unit and/or between two devices such as between a computational device and a further device. In particular, the communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. In particular, the communication interface may provide a data transfer connection, e.g. Bluetooth, NFC, inductive coupling or the like. As an example, the communication interface may be or may comprise at least one port comprising one or more of a network or internet port, a USB-port and a disk drive. The communication interface may be at least one web interface.

The communication interface provides information from and to the processing device. The communication interface may enable transfer of information with at least one input device and/or with at least one output device. The communication interface may enable transfer of information within the processing device. The communication interface may enable transfer of information with a memory of the processing device. The input device may be a physical and/or a logical input device. The physical input device may be or may comprise one or more of at least one keyboard, at least one mouse, at least one touchscreen, at least one touchpad, at least one microphone, at least one gesture-based control, at least one database. The output device may be a physical and/or a logical output device. For example, the physical output device may be or may comprise at least one display and/or at least one monitor. The logical output device may be e.g. an API, a remote-control function, a software function call, an interface to a database. The output device may be comprised by or may be coupled to the communication interface wired or wireless.

For example, the communication interface may comprise at least one web interface configured for providing at least one input box for inputting the at least one physico-chemical property of the oil or the mixture of oils or of the at least one sensory attribute. The web interface may be configured for displaying the determined sensory attribute or the determined physico-chemical property.

The term “processing device” or “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processing device may be configured for processing basic instructions that drive the computer or system. As an example, the processing device may comprise at least one arithmetic logic unit (ALU), at least one floating-point unit (FPU), such as a math coprocessor or a numeric coprocessor, a plurality of registers, specifically registers configured for supplying operands to the ALU and storing results of operations, and a memory, such as an L1 and L2 cache memory. In particular, the processing device may be a multicore processor. Specifically, the processing device may be or may comprise a central processing unit (CPU). Additionally or alternatively, the processing device may be or may comprise a microprocessor, thus specifically the processing device's elements may be contained in one single integrated circuitry (IC) chip. Additionally or alternatively, the processing device may be or may comprise one or more application specific integrated circuits (ASICs) and/or one or more field-programmable gate arrays (FPGAs) or the like. The processing device may be e.g. a general-purpose computer, a CPU, a microprocessor, a FGPA, a network of computers, a network of CPUs.

The term “obtaining” as used herein is a broad term and is to be given its ordinary and customary meaning to a person of ordinary skill in the art and is not to be limited to a special or customized meaning. The term specifically may refer, without limitation, to inputting and/or retrieving the physico-chemical property or the sensory attribute via the communication interface.

The term “physico-chemical property” as used herein is a broad term and is to be given its ordinary and customary meaning to a person of ordinary skill in the art and is not to be limited to a special or customized meaning. The term specifically may refer, without limitation, to at least one measurable physical and/or chemical property of the oil or mixture of oils. As used herein, the term “physico-chemical property of the oil or the mixture of oils” may refer to physico-chemical property of the oil or of different oils of the mixture of oils. Physico-chemical properties may be identified by experimental measurements such as in a laboratory and/or may be provided or retrieved from at least one data sheet or at least one database. For example, physico-chemical properties may be optical properties or mechanical properties. Physico-chemical properties of the oil or the different oils of the mixture may be one or more of density, refractive index, surface tension, interfacial tension, in particular liquid-liquid interfacial tension, spreadability, viscosity, dielectric constant, molecular weight, Equivalent Alkane Carbon Number (EACN) and the like. The correlation between sensory attributes and physico-chemical properties may vary between the physico-chemical properties. Preferably, the physico-chemical properties of the oil or the different oils of the mixture may be at least one of spreadability, and/or viscosity, and/or molecular weight.

The term “refractive index” as used herein is a broad term and is to be given its ordinary and customary meaning to a person of ordinary skill in the art and is not to be limited to a special or customized meaning. The term specifically may refer, without limitation, to a measure of how fast light propagates through oil or a mixture of at least two oils. It is the ratio of speed of light in vacuum divided by speed of light in the product. The refractive index may be measured using a standard device to measure refractive index.

The term “surface tension” as used herein is a broad term and is to be given its ordinary and customary meaning to a person of ordinary skill in the art and is not to be limited to a special or customized meaning. The term specifically may refer, without limitation, to a force that holds a unit length of interface between oil and air. It is typically measured using “Wilhelmy plate method (plate tensiometer)” method.

The term “liquid-liquid interfacial tension” (I FT) as used herein is a broad term and is to be given its ordinary and customary meaning to a person of ordinary skill in the art and is not to be limited to a special or customized meaning. The term specifically may refer, without limitation, to force that holds a unit length of interface between oil and water. It is typically measured using “pendant drop” method. The interfacial tension may be measured in [mN/m] versus water and may be measured using the pendant drop method at a temperature of 23+/−2° C. For example, a Dataphysics OCAH 200 high-speed contact angle measuring system having a cannula (DataPhysics Instruments GmbH, Filderstadt, Germany).

The term “spreadability”, also denoted spreading value, as used herein is a broad term and is to be given its ordinary and customary meaning to a person of ordinary skill in the art and is not to be limited to a special or customized meaning. The term specifically may refer, without limitation, to an area a fixed amount of oil has spread on a collagen surface, in particular substituting human skin, in 10 minutes. This is measured by a method developed in house by Henkel, presently BASF, Düsseldorf.

The term “viscosity”, also denoted spreading value, as used herein is a broad term and is to be given its ordinary and customary meaning to a person of ordinary skill in the art and is not to be limited to a special or customized meaning. The term specifically may refer, without limitation, to flow behavior or rheology of an oil. This may be measured using standard Rheometers.

The dielectric constant may be measured using Qumat 02600 Dekameter device; the EACN method was developed by BASF Düsseldorf [T. H. Förster et al, International Journal of Cosmetic Science, 16, 84-92 (1994)].

The molecular weight may be determined from a chemical composition.

The processing device may be configured for obtaining via the communication interface at least one unique identifier of the oil and/or mixture of oils. The unique identifier for each of the different oils, may be e.g. internal labels, chemical-structure formulas, brand names, CAS number and the like. Using identifiers of oils rather than physico-chemical properties greatly increases usability of the system. The processing device may be configured for retrieving the physico-chemical properties or the sensory attributes for the oil and/or mixture of oils defined by the obtained unique identifier, e.g. from a database.

The processing device may be configured for determining the at least one sensory attribute for the oil and/or mixture of oils defined by the obtained unique identifier based on the retrieved physico-chemical properties and the model. The processing device may be configured for providing via the communication interface the at least one sensory attribute for the oil and/or mixture of oils defined by the obtained unique identifier.

The processing device may be configured for determining the at least one physico-chemical property for the oil and/or mixture of oils defined by the obtained unique identifier based on the retrieved sensory attributes and the model. The processing device may be configured for providing via the communication interface the at least one physico-chemical property for the oil and/or mixture of oils defined by the obtained unique identifier.

The processing device may be configured for changing at least one unique identifier of the different oils such as by receiving a command via the communication interface, e.g. a command entered by a user. By changing at least one unique identifier of the different oils, at least one oil in the mixture is replaced. By changing the at least one unique identifier of the different oils, it may be possible to observe impact of said oil on the cosmetic product and may allow finding a best combination of oils to meet target requirements.

For example, the processing device may be configured for adding an additional unique identifier of the different oils. This may allow quickly determining sensory attributes or physico-chemical properties of new oil mixtures, that are based on more than two oils. In case that target requirements cannot be met by an initial mixture, this may enable to add further oils to the mixture, such that the target requirements can be met.

The model may comprise at least one mathematical algorithm relating one or more physico-chemical properties to one or more sensory attributes. The model may be derived at least partially from data and/or using physico-chemical laws. The model may be derived from statistics (Statistics 4^{th} edition, David Freedman et al., W. W. Norton & Company Inc., 2004). The model may be derived from machine learning (Machine Learning and Deep Learning frameworks and libraries for largescale data mining: a survey, Artificial Intelligence Review 52, 77-124 (2019), Springer). Specifically, the model may be a correlation model. The model may describe the relation between one or more physico-chemical properties to one or more sensory attributes as a function. Correlation between one or more physico-chemical properties to one or more sensory attributes may be determined by experiments. The model may be configured for evaluating from one or more physicochemical properties the corresponding sensory attribute(s). For example, the model may comprise correlation functions between one or more of viscosity and grease, viscosity and thickness, and/or spreading value and thickness of residue in the form of

grease=a viscosity+b;

thickness=a⋅viscosity²+b⋅viscosity+c

thickness of residue=−a⋅spreading value²−b⋅spreading value+c,

wherein a, b and c are constants which were determined by fitting measurement results such as respective correlation plots for viscosity and grease, viscosity and thickness, and/or spreading value and thickness of residue. The processing device may apply the obtained physico-chemical property or the obtained sensory attribute to the model and determines the related sensory attributes or the related physico-chemical properties, such as by using the correlations as outlined above. The determined sensory attribute may comprise sensory attributes for each of the oils. The determined physico-chemical property may comprise physico-chemical properties for each of the oils.

For example, for oils the following correlation functions may be used:

grease=0.566+0.07 viscosity,

thickness=−7.22×10⁻⁵ viscosity²+1.366×10⁻² viscosity+0.8566,

thickness of residue=1.21×10⁻⁸ spreadability²−1.837×10⁻⁴ spreadability+1.24.

It was found that for mixtures of oils the same correlation functions are suitable.

Sensory attributes and/or physico-chemical properties of a mixture of oils can be determined based on a data driven model and/or a rigorous model and composition parameters of the mixture of oils. Specifically, performance properties such as sensory attributes and/or physicochemical properties of a mixture of oils can be determined, e.g. by using the processing device, as described in WO 2021/180922, the content of which is included herein by reference. The performance properties for each of the different oils may be a plurality of performance properties for each of the different oils. The performance properties may be any combination of physico-chemical properties. The performance properties may be any combination of sensory properties. The performance properties may be any combination of physico-chemical and sensory properties. The data driven model may describe the relation between the performance properties of each of the oils in the mixture and the measure for the ratio of the different oils in the mixture and the performance properties of the mixture. The data driven model maybe based on measurements of performance properties of different oils in mixtures and a measure for the ratio of the different oils in these mixtures. The data driven model describes the relation between the performance properties of each of the surfactants and/or further components in the mixture and the measure for the ratio of the different surfactants and/or further components in the mixture and the performance properties of the mixture. The data driven model maybe on measurements of performance properties of surfactants and further components in mixtures and a measure for the ratio of the surfactants and further components in these mixtures. The data driven model maybe on measurements of performance properties of different oils and/or surfactants in mixtures and a measure for the ratio of the different oils and/or for the ratio of the surfactants in these mixtures. The data driven model may refer to a model at least partially derived from data. In contrast to a rigorous model that is purely derived using physico-chemical laws. Use of a data driven model can allow describing relations, that cannot be modelled by physicochemical laws. The use of data driven models can allow to describe relations without solving equations from physico-chemical laws. This can reduce computational power. This can improve speed. The data driven model may be derived from statistics (Statistics 4^{th} edition, David Freedman et al., W. W. Norton & Company Inc., 2004). The data driven model may be derived from machine learning (Machine Learning and Deep Learning frameworks and libraries for largescale data mining: a survey, Artificial Intelligence Review 52, 77-124 (2019), Springer). The data driven model may be a regression model. The data driven model may be a mathematical model. The mathematical model may describe the relation between provided performance properties and determined performance properties as a function. The data driven model may be any other machine learning model. The data driven model may be a machine learning model. The data driven model may be trained based on one or more of “historic” composition parameters, “historic performance properties” or quantum mechanical descriptors such as described in C. C. Pye, T. Ziegler, E. van Lenthe, J. N. Louwen, Can. J. Chem. 87, 790 (2009).

For example, the data driven model is a linear mixing model. Other examples, however, are possible, e.g. the data driven model may be a log-log model. For example, the performance properties of the mixture are determined with the processing device, based on the data driven model and/or the rigorous model and the composition parameters. The linear model in this example can be described by SFT(mixture₂)=Σ₁² SFT (oilᵢ)·xᵢ. “SFT” may be surface tension. The data driven model in this example was derived by linear regression on measurements of different ratios. Index 2 indicates that the mixture comprises two different oils. The term xᵢ. relates to the relative portion of oil, in the mixture in percent. When the performance properties comprise more than one performance property for each of the different oils the performance of each oil may be provided as a vector \vec{prop}(oilᵢ) for oil i. For the example given in table 1, the properties are SFT, RI, spreading value and density. And the mixing ratio x₁ is 25% and x₂=100−25=75%.

TABLE 1

| | Oil₁ | Oil₂ | | Performance | Performance |
|---|---|---|---|---|---|
| Performance | (Cetiol | (Cetiol | | properties | properties |
| property | OE) | LC) | x₁ | determined | measured |
| SFT | 27.26 | 29.80 | 25 | 29.16 | |
| (mN/m) | | | | | |
| RI | 1.433 | 1.445 | 25 | 1.442 | 1.442 |
| Spreading value | 1607 | 557 | 25 | 819 | 729 |
| (mm²/10 min) | | | | | |
| Density | 0.805 | 0.856 | 25 | 0.8432 | 0.8427 |
| (gm/cm³) | | | | | |

The performance property vector for the example in table 1 would therefore look like

$$p \vec{r} o p = ( \begin{matrix}\begin{matrix}\begin{matrix}SFT \\ RI\end{matrix} \\ spreading value\end{matrix} \\ density\end{matrix} )$$

For a mixture of two oils the step of determination of the determined properties of the mixture could be described in a general way as:

\vec{prop}(mixtureᵢⱼ)=f[\vec{prop}(oilᵢ), \vec{prop}(oilⱼ), xᵢⱼ]

with xᵢⱼ: mixing ratio of the two different oils and \vec{prop}(oilᵢ): the vector with performance properties of oil/and \vec{prop}(mixtureᵢⱼ): the vector with performance properties of the oil mixture. The function f is the mathematical description of the data driven model.

An example of a linear data driven model for a multitude of n oils is described by the formula below.

$$\vec{p r o p} ( {mixture}_{ij … n} ) = {\sum }_{i}^{n} \vec{p r o p} ( {oil}_{i} ) \cdot {x}_{i}$$

For example, the system may be configured for determining the physico-chemical properties of the mixture considering the components of the mixture as described above. The determined physico-chemical properties of the mixture may be used as input for the model for determining the at least one sensory attribute of the mixture.

For example, the system may be configured for determining the sensory attribute of the mixture considering the components of the mixture as described above. The determined sensory attribute of the mixture may be used as input for the model for determining the at least one physicochemical property of the mixture.

As the person skilled in the art will understand, the determining of the sensory attribute or physicochemical properties using the model according to the present invention may be performed for correlated sensory attributes and physico-chemical properties. Additionally, lesser or even non-correlated sensory attributes and physico-chemical properties may be used, e.g. as additional constrains.

For example, the processing device may be configured for obtaining via the communication interface at least one target sensory attribute. The term “target sensory attribute” as used herein is a broad term and is to be given its ordinary and customary meaning to a person of ordinary skill in the art and is not to be limited to a special or customized meaning. The term specifically may refer, without limitation, to at least one demanded and/or required sensory attribute of the personal care product. For example, the target sensory attribute may be a sensory attribute a developer or consumer may ask for. The processing device may be configured for determining at least one physico-chemical property based on the obtained sensory attributes and the model. The processing device may be configured for determining a target oil or a target ratio of a mixture of oils having the obtained target sensory attribute based on the determined physico-chemical property.

For example, the processing device may be configured for obtaining via the communication interface at least one target physico-chemical property. The term “target physico-chemical property” as used herein is a broad term and is to be given its ordinary and customary meaning to a person of ordinary skill in the art and is not to be limited to a special or customized meaning. The term specifically may refer, without limitation, to at least one demanded and/or required physico-chemical property of the personal care product. For example, the target physico-chemical property may be a physico-chemical property a developer or consumer may ask for. The processing device may be configured for determining at least one sensory attribute based on the obtained physicochemical property and the model. The processing device may be configured for determining a target oil or a target ratio of a mixture of oils having the obtained target sensory attribute based on the determined sensory attribute.

For example, the target sensory attribute or the target physico-chemical property may be properties of a known/real oil or oil mixture. This may for example occur, when an undesired oil is sought to be replaced. Oils may be undesired if they are not environmentally friendly. Oils may be undesired if they do not have approval for use in cosmetics. The target physico-chemical property or target sensory attribute for a specific oil or for a mixture of oils may be a requirement for a new formulation or a new mixture of oils. This may occur, when a new formulation needs to be designed such that certain target requirements are met. In such cases target requirements may be provided by a customer for example based on market research.

For example, the oil or the mixture of oils may comprise a mineral oil or a mixture containing at least one mineral/paraffin oil. Throughout this application paraffin oils are mineral oils. Paraffin oils are very common in personal care products due to their valued sensory properties. However, despite their advantages in user experience, they are not considered sustainable. For example, the oil or the mixture of oils may comprise a silicone-based oil. Silicone-based oils are very common in personal care products due to their valued sensory properties. Despite their advantages in user experience, they are not considered sustainable. The system according to the present invention allows to replace the mineral/paraffin based oil or oils or silicone-based oil or oils with an oil or oils that are more environmentally friendly. For example, the environmentally friendly oil(s) constitute natural raw ingredients. The environmentally friendly oil(s) may be biodegradable.

The processing device may be configured for determining a composition of a cosmetic product comprising the oil and/or mixture of oils based on the provided physico-chemical property or the provided sensory attribute. The cosmetic product may be a personalized cosmetic product. The term “personalized cosmetic product” as used herein is a broad term and is to be given its ordinary and customary meaning to a person of ordinary skill in the art and is not to be limited to a special or customized meaning. The term specifically may refer, without limitation, to a cosmetic product which is tailored to accommodate at least one specific individual or a group of specific individuals.

The system may comprise at least one testing unit configured for testing at least one physicochemical property and/or at least one sensory attribute of an oil or mixture of oils, thereby determining test results. For example, the testing unit may be configured for testing at least one physico-chemical property and/or at least one sensory attribute of the cosmetic product comprising the oil and/or mixture of oils having the physico-chemical property or the sensory attribute determined by the processing device. Alternatively, the communication interface may be configured for retrieving test results such as from at least one laboratory. The processing device may be configured for comparing the target physico-chemical property or target sensory attribute with the test results of the cosmetic product and deriving a result of the comparison. Specifically, the processing device may be configured for determining if the test results meet the target physicochemical property or target sensory attribute at least within tolerances. This may allow determining if a specific oil or specific mixture of oils can be replaced. The processing device may be configured for adapting the model and providing the adapted model.

The determined sensory attribute or the determined physico-chemical property may be used as control data, e.g. within a production chain. Additionally or alternatively, the formulation relating to the sensory attribute or the determined physico-chemical property may be used as control data, e.g. within a production chain. The system may be configured for issuing at least one indication in case a deviation of the at least one test result and the target physico-chemical property or target sensory attribute exceeds a pre-defined tolerance range. The indication may comprise at least one warning. The system may be configured for changing the used oil and/or at least one oil of the mixture and/or a ratio of the oils of the mixture of oils in case a deviation of the at least one test result and the target physico-chemical property or target sensory attribute exceeds a predefined tolerance range.

The system may further comprise a mixing module configured for controlling mixing the personal care product, in particular cosmetics. The determined sensory attribute or the determined physico-chemical property may be used as control data for the mixing module. Additionally or alternatively, the formulation relating to the sensory attribute or the determined physico-chemical property may be used as control data for the mixing module.

In a further aspect, a computer-implemented method for predicting sensory attributes or physicochemical properties of an oil or a mixture of oils for personal care by using at least one system according to the present invention is proposed. With respect to terms, definitions and embodiments reference is made to the description of the system.

The term “computer-implemented” as used herein is a broad term and is to be given its ordinary and customary meaning to a person of ordinary skill in the art and is not to be limited to a special or customized meaning. The term specifically may refer, without limitation, to a process which is fully or partially implemented by using a data processing means, such as data processing means comprising at least one processing device. The term “computer”, thus, may generally refer to a device or to a combination or network of devices having at least one data processing means such as at least one processing device. The computer, additionally, may comprise one or more further components, such as at least one of a data storage device, an electronic interface or a human-machine interface.

The computer-implemented method comprises the following steps, which may be performed in the given order. However, a different order may also be possible. Further, one or more than one or even all of the steps may be performed once or repeatedly. Further, the method steps may be performed in a timely overlapping fashion or even in parallel. The method may further comprise additional method steps which are not listed.

The method comprises the following steps:
a) obtaining via the communication interface of at least one physico-chemical property of the oil or the mixture of oils or of at least one sensory attribute and of at least one model relating one or more physico-chemical properties to one or more sensory attributes by using the processing device;
b) determining at least one sensory attribute of the oil or the mixture of oils based on the obtained physico-chemical properties and the model or determining at least one physicochemical property of the oil or the mixture of oils based on the obtained sensory attributes and the model by using the processing device;
c) providing via the communication interface the determined sensory attribute or the determined physico-chemical property by using the processing device.

The providing via the communication interface may further comprise providing composition parameters and/or a formulation of the mixture, and/or a formulation of the oil-containing product for cosmetics.

In a further aspect a computer program for performing the method according to the present invention is proposed. The computer program comprises instructions which, when the program is executed by a computer or a computer network, cause the computer or the computer network to fully or partially perform the method according to the present invention in one or more of the embodiments enclosed herein. For possible definitions of most of the terms used herein, reference may be made to the description of the system and the method above or as described in further detail below. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps a) to c) as indicated above may be performed by using a computer or a computer network, preferably by using a computer program.

Further disclosed and proposed herein is a computer program product having program code means, in order to perform the method according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In a further aspect, a use of the determined sensory attribute or the determined physico-chemical property determined according to the method according to the present invention for producing a personal care product is proposed. With respect to terms, definitions and embodiments reference is made to the description of the method and system.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically are used only once when introducing the respective feature or element. In most cases, when referring to the respective feature or element, the expressions “at least one” or “one or more” are not repeated, non-withstanding the fact that the respective feature or element may be present once or more than once.

Further, as used herein, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In another aspect disclosed herein is a computer-implemented method for monitoring and/or validating production of a personal care product comprising a mixture of oils, the method comprising the steps of:
receiving the sensory attributes as generated according to any of the methods disclosed herein,
monitoring and/or validating the personal care product based on the sensory attributes.

In another aspect disclosed herein is a computer-implemented method for monitoring production of a personal care product comprising a mixture of oils, the method comprising the steps of:
receiving target sensory attributes in particular sensory attributes as generated according to any of the methods disclosed herein, wherein the sensory attributes are indicative of quality criteria,
measuring one or more physico-chemical properties of the produced personal care product,
determining, sensory attributes of the produced personal care product based on the measured physico-chemical properties
determining based on the determined sensory attributes and the target sensory attributes, if the produced personal care product fulfils quality criteria.

In another aspect disclosed herein is an apparatus for monitoring and/or validating production of a personal care product, the apparatus comprising one or more processing unit(s) configured to monitor and/or validate production, wherein the processing unit(s) include instructions, which when executed on the one or more processing unit(s) perform the following steps:
receiving the sensory attributes as generated/predicted according to any of the methods disclosed herein,
monitoring and/or validating the fragrance product based on the sensory attributes.

In another aspect disclosed herein is an apparatus for monitoring production of a personal care product, the apparatus comprising one or more processing unit(s) configured to monitor production, wherein the processing unit(s) include instructions, which when executed on the one or more processing unit(s) perform the following steps:
receiving target sensory attributes in particular as generated according to any of the methods disclosed herein, wherein the sensory attributes are indicative of quality criteria,
measuring physical properties of the produced personal care product,
determining sensory attributes of the produced personal care product based on the measured physico-chemical properties,
determining, based on the determined sensory attributes and the target sensory attributes, if the produced fragrance product fulfils quality criteria.

In another aspect disclosed herein is an apparatus for validating production of a personal care product, the apparatus comprising one or more processing unit(s) configured to validate production of a fragrance product, wherein the processing unit(s) include instructions, which when executed on the one or more processing unit(s) perform the following steps:
receiving an existing sensory profile, in particular as generated according to any of the methods disclosed herein, preferably based on existing personal care products associated with existing ingredients, wherein the existing sensory attributes are indicative of quality criteria,
receiving physico-chemical properties for a new personal care product associated with at least one new ingredient and generating sensory attributes profile according to any of the methods disclosed herein based on the new ingredient data,
determining, based on the existing sensory attributes and the new sensory attributes profile, if the new ingredient(s) fulfils quality criteria.

Summarizing and without excluding further possible embodiments, the following embodiments may be envisaged:

Embodiment 1. System for predicting sensory attributes or physico-chemical properties of an oil or a mixture of oils for personal care, wherein the system comprises at least one communication interface for providing data and at least one processing device, wherein the processing device is configured for:
obtaining via the communication interface of at least one physico-chemical property of the oil or the mixture of oils or of at least one sensory attribute and of at least one model relating one or more physico-chemical properties to one or more sensory attributes;
determining at least one sensory attribute of the oil or the mixture of oils based on the obtained physico-chemical properties and the model or determining at least one physico-chemical property of the oil or the mixture of oils based on the obtained sensory attributes and the model;
providing via the communication interface the determined sensory attribute or the determined physico-chemical property.

Embodiment 2. System for predicting sensory attributes of an oil or a mixture of oils for personal care, wherein the system comprises at least one communication interface for providing data and at least one processing device, wherein the processing device is configured for:
obtaining via the communication interface of at least one physico-chemical property of the oil or the mixture of oils and of at least one model relating one or more physico-chemical properties to one or more sensory attributes;
determining at least one sensory attribute of the oil or the mixture of oils based on the obtained physico-chemical properties and the model;
providing via the communication interface the determined sensory attribute.

Embodiment 3. System for predicting physico-chemical properties of an oil or a mixture of oils for personal care, wherein the system comprises at least one communication interface for providing data and at least one processing device, wherein the processing device is configured for:
obtaining via the communication interface of at least one sensory attribute and of at least one model relating one or more physico-chemical properties to one or more sensory attributes;
determining at least one physico-chemical property of the oil or the mixture of oils based on the obtained sensory attributes and the model;
providing via the communication interface the determined physico-chemical property.

Embodiment 4. The system according to any one of the preceding embodiments, wherein the mixture of oils comprises at least two different oils, at least three different oils or at least four different oils.

Embodiment 5. The system according to any one of the preceding embodiments, wherein the determined sensory attribute comprises sensory attributes for each of the oils.

Embodiment 6. The system according to any one of the preceding embodiments, wherein the determined physico-chemical property comprises physico-chemical properties for each of the oils.

Embodiment 7. The system according to any one of the preceding embodiments, wherein the processing device is configured for obtaining via the communication interface at least one unique identifier of the oil and/or mixture of oils, wherein the processing device is configured for retrieving the physico-chemical properties or the sensory attributes for the oil and/or mixture of oils defined by the obtained unique identifier.

Embodiment 8. The system according to the preceding embodiment, wherein the processing device is configured for determining the at least one sensory attribute for the oil and/or mixture of oils defined by the obtained chemical formulation and/or the unique identifier based on the retrieved physico-chemical properties and the model, wherein the processing device is configured for providing via the communication interface the at least one sensory attribute for the oil and/or mixture of oils defined by the obtained chemical formulation and/or the unique identifier.

Embodiment 9. The system according to any one of the preceding embodiments, wherein the processing device is configured for obtaining via the communication interface at least one target sensory attribute, wherein the processing device is configured for determining at least one physico-chemical property based on the obtained sensory attributes and the model, wherein the processing device is configured for determining a target oil or a target ratio of a mixture of oils having the obtained target sensory attribute based on the determined physicochemical property.

Embodiment 10. The system according to any one of the preceding embodiments, wherein the processing device is configured for obtaining via the communication interface at least one target physico-chemical property, wherein the processing device is configured for determining at least one sensory attribute based on the obtained physico-chemical property and the model, wherein the processing device is configured for determining a target oil or a target ratio of a mixture of oils having the obtained target physico-chemical property based on the determined sensory attribute.

Embodiment 11. The system according to any one of the preceding embodiments, wherein the processing device is configured for determining a composition of a cosmetic product comprising the oil and/or mixture of oils based on the provided physico-chemical property or the provided sensory attribute.

Embodiment 12. The system according to the preceding embodiment, wherein the cosmetic product is a personalized cosmetic product.

Embodiment 13. The system according to any one of the preceding embodiments, wherein the communication interface comprises at least one web interface configured for providing at least one input box for inputting the at least one physico-chemical property of the oil or the mixture of oils or of the at least one sensory attribute.

Embodiment 14. The system according to the preceding embodiment, wherein the web interface is configured for displaying the determined sensory attribute or the determined physico-chemical property.

Embodiment 15. A computer implemented method for predicting sensory attributes or physicochemical properties of an oil or a mixture of oils for personal care by using at least one system according to any one of the preceding embodiments, wherein the method comprises the following steps:
a) obtaining via the communication interface of at least one physico-chemical property of the oil or the mixture of oils or of at least one sensory attribute and of at least one model relating one or more physico-chemical properties to one or more sensory attributes by using the processing device;
b) determining at least one sensory attribute of the oil or the mixture of oils based on the obtained physico-chemical properties and the model or determining at least one physico-chemical property of the oil or the mixture of oils based on the obtained sensory attributes and the model by using the processing device;
c) providing via the communication interface the determined sensory attribute or the determined physico-chemical property by using the processing device.

Embodiment 16. A computer program including computer-executable instructions for performing the method according to the preceding embodiment when the program is executed on a computer or computer network.

Embodiment 17. A computer program product having program code means, in order to perform the method according to embodiment 15 when the program is executed on a computer or computer network.

Embodiment 18. Use of the determined sensory attribute or the determined physico-chemical property determined according to embodiment 15 for producing a personal care product.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows an example of a system for predicting sensory attributes or physico-chemical properties according to the present invention;

FIG. 2 shows an example method/flow-chart for a computer-implemented method for predicting sensory attributes or physico-chemical properties according to the present invention;

FIGS. 3A to 3D show exemplary correlation plots and table;

FIGS. 4A to 4F shows further exemplary correlations plots;

FIG. 5 shows an example of a flowchart for monitoring and/or controlling quality of a personal care product comprising a mixture of oils;

FIG. 6 shows an example of the flowchart for validating the production of a personal care product comprising a mixture of oils with at least one new ingredient based on the physico-chemical properties personal care product;

FIG. 7 shows an example of a production line for producing the personal care product with a monitoring apparatus; and

FIG. 8 shows an example of a production line for producing the personal care product with a validation apparatus.

### DETAILED DESCRIPTION OF THE EMBODIMENTS

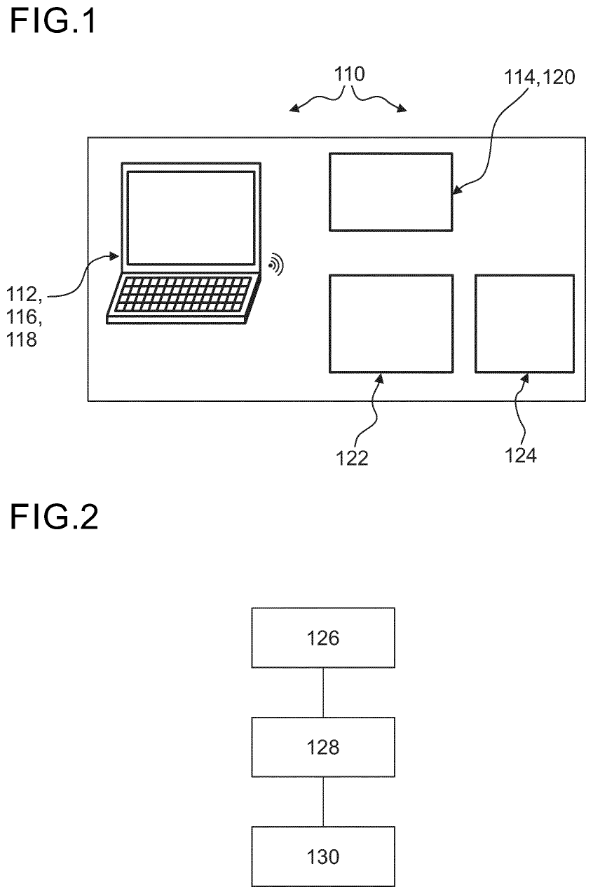

FIG. 1 shows an example of a system 110 for predicting sensory attributes or physico-chemical properties according to the present invention. The system 110 comprises at least one communication interface 112 for providing data and at least one processing device 114. The processing device 114 is configured for:
obtaining via the communication interface 112 of at least one physico-chemical property of the oil or the mixture of oils or of at least one sensory attribute and of at least one model relating one or more physico-chemical properties to one or more sensory attributes;
determining at least one sensory attribute of the oil or the mixture of oils based on the obtained physico-chemical properties and the model or determining at least one physicochemical property of the oil or the mixture of oils based on the obtained sensory attributes and the model;
providing via the communication interface 112 the determined sensory attribute or the determined physico-chemical property.
The communication interface 112 may be configured for transferring information from a computational device, e.g. a computer, such as to send or output information, e.g. onto another device. Additionally or alternatively, the communication interface 112 may be configured for transferring information onto a computational device, e.g. onto a computer, such as to receive information. The communication interface 112 may specifically provide means for transferring or exchanging information. In particular, the communication interface 112 may provide a data transfer connection, e.g. Bluetooth, NFC, inductive coupling or the like. As an example, the communication interface may be or may comprise at least one port comprising one or more of a network or internet port, a USB-port and a disk drive. The communication interface 112 may be at least one web interface.

The communication interface 112 provides information from and to the processing device 114. The communication interface 112 may enable transfer of information with at least one input device 116 and/or with at least one output device 118. The communication interface 112 may enable transfer of information within the processing device 114. The communication interface 112 may enable transfer of information with a memory 120 of the processing device 114. The input device 116 may be a physical and/or a logical input device. The physical input device may be or may comprise one or more of at least one keyboard, at least one mouse, at least one touchscreen, at least one touchpad, at least one microphone, at least one gesture-based control, at least one database. The output device 118 may be a physical and/or a logical output device. For example, the physical output device may be or may comprise at least one display and/or at least one monitor. The logical output device may be e.g. an API, a remote-control function, a software function call, an interface to a database. The output device 118 may be comprised by or coupled to the communication interface 112 wired or wireless.

For example, the communication interface 112 may comprise at least one web interface configured for providing at least one input box for inputting the at least one physico-chemical property of the oil or the mixture of oils or of the at least one sensory attribute. The web interface may be configured for displaying the determined sensory attribute or the determined physico-chemical property.

The processing device 114 may be an arbitrary logic circuitry configured for performing basic operations of a computer or system, and/or, generally, to a device which is configured for performing calculations or logic operations. In particular, the processing device 114 may be configured for processing basic instructions that drive the computer or system. As an example, the processing device 114 may comprise at least one arithmetic logic unit (ALU), at least one floating-point unit (FPU), such as a math coprocessor or a numeric coprocessor, a plurality of registers, specifically registers configured for supplying operands to the ALU and storing results of operations, and the memory 120, such as an L1 and L2 cache memory. In particular, the processing device 114 may be a multicore processor. Specifically, the processing device 114 may be or may comprise a central processing unit (CPU). Additionally or alternatively, the processing device 114 may be or may comprise a microprocessor, thus specifically the processing device's elements may be contained in one single integrated circuitry (IC) chip. Additionally or alternatively, the processing device may be or may comprise one or more application specific integrated circuits (ASICs) and/or one or more field-programmable gate arrays (FPGAs) or the like. The processing device 114 may be e.g. a general-purpose computer, a CPU, a microprocessor, a FGPA, a network of computers, a network of CPUs.

The obtaining may comprise inputting and/or retrieving the physico-chemical property or the sensory attribute via the communication interface 112.

Physico-chemical properties may be identified by experimental measurements such as in a laboratory and/or may be provided or retrieved from at least one data sheet or at least one database. For example, physico-chemical properties may be optical properties or mechanical properties. Physico-chemical properties of the oil or the different oils of the mixture may be one or more of density, refractive index, surface tension, interfacial tension, in particular liquid-liquid interfacial tension, spreadability, viscosity, dielectric constant, molecular weight, Equivalent Alkane Carbon Number (EACN) and the like.

The sensory attribute may be or may comprise features characterizing perception of the oil or the mixture by human senses when used in the respective field of use. In particular, sensory attributes may determine how a product for personal care comprising the oil or oil mixture, in particular cosmetics comprising the oil or oil mixture, is perceived on human skin. Sensory attributes may comprise at least one attribute selected from the group consisting of: wetness, spreadability, thickness, absorption, distribution, oily, greasy, amount of residue, slipperiness, smoothness, stickiness, dryness, gloss, and the like. These properties may be classified into those measured immediately after application, in rub-out phase and after N minutes after application. Here N can be but is not limited to 5 minutes, 20 minutes, etc. The above-mentioned examples are beneficial as they are well suited to describe the user experience of a cosmetic product. The individual sensory attributes are self-explanatory in accordance to the respective names. Sensory attributes may be defined differently in different labs.

The processing device 114 may be configured for obtaining via the communication interface 112 at least one unique identifier of the oil and/or mixture of oils. The unique identifier for each of the different oils, may be e.g. internal labels, chemical-structure formulas, brand names, CAS number and the like. Using identifiers of oils rather than physico-chemical properties greatly increases usability of the system 110. The processing device 114 may be configured for retrieving the physico-chemical properties or the sensory attributes for the oil and/or mixture of oils defined by the obtained unique identifier, e.g. from a database.

The processing device 114 may be configured for determining the at least one sensory attribute for the oil and/or mixture of oils defined by the obtained unique identifier based on the retrieved physico-chemical properties and the model. The processing device 114 may be configured for providing via the communication interface 112 the at least one sensory attribute for the oil and/or mixture of oils defined by the obtained unique identifier.

The processing device 114 may be configured for determining the at least one physico-chemical property for the oil and/or mixture of oils defined by the obtained unique identifier based on the retrieved sensory attributes and the model. The processing device 114 may be configured for providing via the communication interface 112 the at least one physico-chemical property for the oil and/or mixture of oils defined by the obtained unique identifier.

The processing device 114 may be configured for changing at least one unique identifier of the different oils such as by receiving a command via the communication interface 112, e.g. a command entered by a user. By changing at least one unique identifier of the different oils, at least one oil in the mixture is replaced. By changing the at least one unique identifier of the different oils, it may be possible to observe impact of said oil on the cosmetic product and may allow finding a best combination of oils to meet target requirements.

For example, the processing device 114 may be configured for adding an additional unique identifier of the different oils. This may allow quickly determining sensory attributes or physico-chemical properties of new oil mixtures, that are based on more than two oils. In case that target requirements cannot be met by an initial mixture, this may enable to add further oils to the mixture, such that the target requirements can be met.

The model may comprise at least one mathematical algorithm relating one or more physico-chemical properties to one or more sensory attributes. The model may be derived at least partially from data and/or using physico-chemical laws. The model may be derived from statistics (Statistics 4^{th} edition, David Freedman et al., W. W. Norton & Company Inc., 2004). The model may be derived from machine learning (Machine Learning and Deep Learning frameworks and libraries for largescale data mining: a survey, Artificial Intelligence Review 52, 77-124 (2019), Springer). Specifically, the model may be a correlation model. The model may describe the relation between one or more physico-chemical properties to one or more sensory attributes as a function. Correlation between one or more physico-chemical properties to one or more sensory attributes may be determined by experiments. The model may be configured for evaluating from one or more physicochemical properties the corresponding sensory attribute(s).

For example, the model may comprise correlation functions between one or more of viscosity and grease, viscosity and thickness, and/or spreading value and thickness of residue in the form of

grease=a viscosity+b;

thickness=a⋅viscosity²+b⋅viscosity+c

thickness of residue=−a⋅spreading value²−b⋅spreading value+c,

wherein a, b and c are constants which were determined by fitting measurement results such as respective correlation plots for viscosity and grease, viscosity and thickness, and/or spreading value and thickness of residue. The processing device 114 may apply the obtained physicochemical property or the obtained sensory attribute to the model and determines the related sensory attributes or the related physico-chemical properties, such as by using the correlations as outlined above. The determined sensory attribute may comprise sensory attributes for each of the oils. The determined physico-chemical property may comprise physico-chemical properties for each of the oils.

For example, the processing device 114 may be configured for obtaining via the communication interface 112 at least one target sensory attribute. For example, the target sensory attribute may be a sensory attribute a developer or consumer may ask for. The processing device 114 may be configured for determining at least one physico-chemical property based on the obtained sensory attributes and the model. The processing device 114 may be configured for determining a target oil or a target ratio of a mixture of oils having the obtained target sensory attribute based on the determined physico-chemical property.

For example, the processing device 114 may be configured for obtaining via the communication interface 112 at least one target physico-chemical property. For example, the target physico-chemical property may be a physico-chemical property a developer or consumer may ask for. The processing device 114 may be configured for determining at least one sensory attribute based on the obtained physico-chemical property and the model. The processing device 114 may be configured for determining a target oil or a target ratio of a mixture of oils having the obtained target sensory attribute based on the determined sensory attribute.

For example, the target sensory attribute or the target physico-chemical property may be properties of a known/real oil or oil mixture. This may for example occur, when an undesired oil is sought to be replaced. Oils may be undesired if they are not environmentally friendly. Oils may be undesired if they do not have approval for use in cosmetics. The target physico-chemical property or target sensory attribute for a specific oil or for a mixture of oils may be a requirement for a new formulation or a new mixture of oils. This may occur, when a new formulation needs to be designed such that certain target requirements are met. In such cases target requirements may be provided by a customer for example based on market research.

For example, the oil or the mixture of oils may comprise a mineral oil or a mixture containing at least one mineral/paraffin oil. Throughout this application paraffin oils are mineral oils. Paraffin oils are very common in personal care products due to their valued sensory properties. However, despite their advantages in user experience, they are not considered sustainable. For example, the oil or the mixture of oils may comprise a silicone-based oil. Silicone-based oils are very common in personal care products due to their valued sensory properties. Despite their advantages in user experience, they are not considered sustainable. The system 110 according to the present invention allows to replace the mineral/paraffin based oil or oils or silicone-based oil or oils with an oil or oils that are more environmentally friendly. For example, the environmentally friendly oil(s) constitute natural raw ingredients.

The processing device 114 may be configured for determining a composition of a cosmetic product comprising the oil and/or mixture of oils based on the provided physico-chemical property or the provided sensory attribute. The cosmetic product may be a personalized cosmetic product.

The system 110 may comprise at least one testing unit 122 configured for testing at least one physico-chemical property and/or at least one sensory attribute of an oil or mixture of oils, thereby determining test results. For example, the testing unit 122 may be configured for testing at least one physico-chemical property and/or at least one sensory attribute of the cosmetic product comprising the oil and/or mixture of oils having the physico-chemical property or the sensory attribute determined by the processing device. Alternatively, the communication interface 112 may be configured for retrieving test results such as from at least one laboratory. The processing device 114 may be configured for comparing the target physico-chemical property or target sensory attribute with the test results of the cosmetic product and deriving a result of the comparison. Specifically, the processing device may be configured for determining if the test results meet the target physico-chemical property or target sensory attribute at least within tolerances. This may allow determining if a specific oil or specific mixture of oils can be replaced. The processing device 114 may be configured for adapting the model and providing the adapted model.

The system 110 may further comprise a mixing module 124 configured for controlling mixing the personal care product, in particular cosmetics. Some models relating physico-chemical properties to one or more sensory attributes and vice-versa may be comprised by the mixing module 124.

FIG. 2 shows an example method/flow-chart for a computer-implemented method for predicting sensory attributes or physico-chemical properties.

The method comprises the following steps:
a) (denoted with reference number 126) obtaining via the communication interface 112 of at least one physico-chemical property of the oil or the mixture of oils or of at least one sensory attribute and of at least one model relating one or more physico-chemical properties to one or more sensory attributes by using the processing device 114;
b) (denoted with reference number 128) determining at least one sensory attribute of the oil or the mixture of oils based on the obtained physico-chemical properties and the model or determining at least one physico-chemical property of the oil or the mixture of oils based on the obtained sensory attributes and the model by using the processing device 114;
c) (denoted with reference number 130) providing via the communication interface the determined sensory attribute or the determined physico-chemical property by using the processing device 114.

Step a) may comprise determining the at least one sensory attribute and/or the at least one physico-chemical property of a mixture of oils based on a data driven model and/or a rigorous model and composition parameters of the mixture of oils, e.g. as described above or in WO 2021/180922, the content of which is included herein by reference.

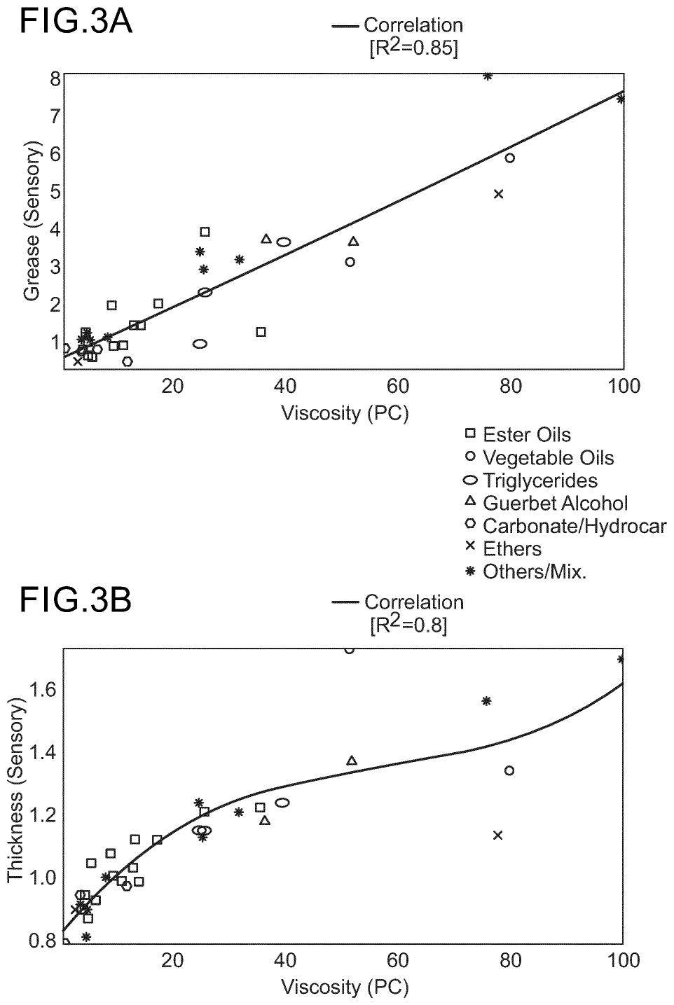

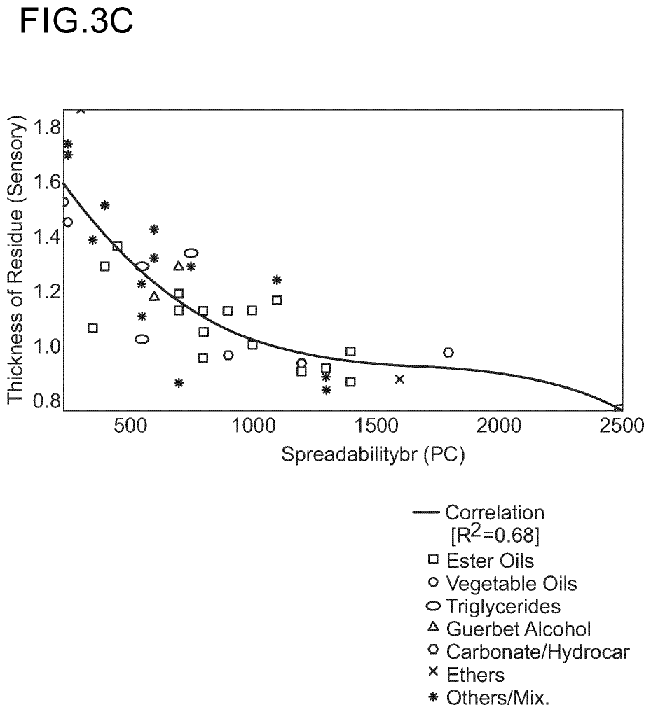

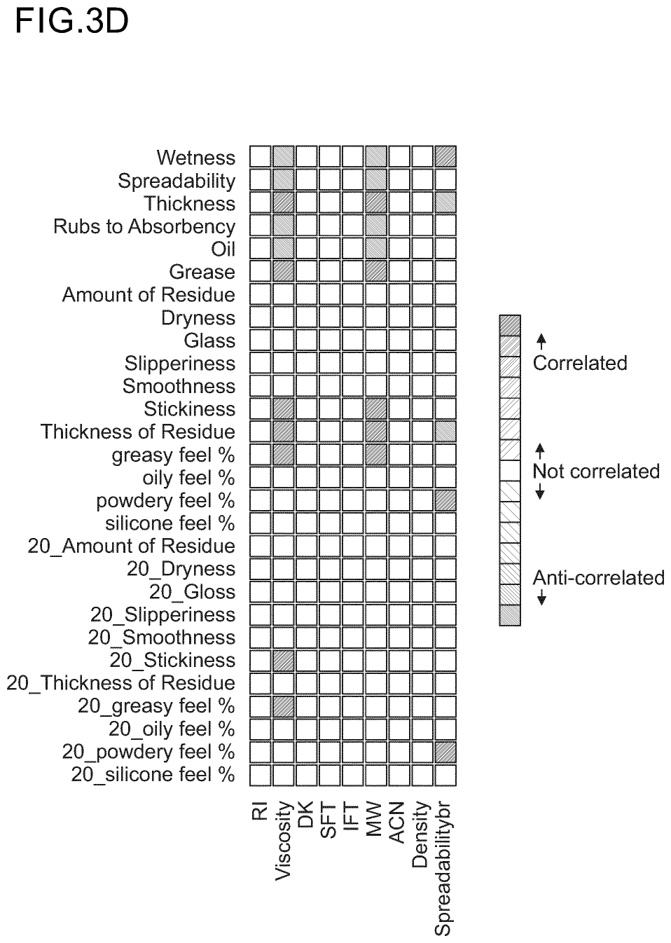

FIGS. 3A to 3C shows experimental results of correlation plots. FIG. 3A shows the correlation between the sensory attribute (“sensory”) grease and the physico-chemical property (“PC”) viscosity for ester oils, vegetable oils, triglycerides, guerbert alcohol, carbonate/hydrocar, ethers and others/mix. In addition, a linear fit is shown. FIG. 3B shows the correlation between the sensory attribute thickness and the physico-chemical property viscosity for ester oils, vegetable oils, triglycerides, guerbert alcohol, carbonate/hydrocar, ethers and others/mix. In addition, a polynominal fit is shown. FIG. 3C shows the correlation between the sensory attribute thickness of residue and the physico-chemical property spreadability (denoted “spreadabilitybr”) for ester oils, vegetable oils, triglycerides, guerbert alcohol, carbonate/hydrocar, ethers and others/mix. In addition, a polynomial fit is shown. Each individual figure illustrates that if the PC property of an oil/oil mixture is known, the corresponding sensory attribute can be evaluated from the particular correlation model. The exact form of such correlation models are described in the equations above. FIG. 3D shows the correlation table between sensory attributes on the y axis and physico-chemical properties on the x axis. “RI” may be the refractive index, “SFT” may be surface tension, “IFT” may be interfacial tension, “MW” may be molecular weight and“ACN” may be Alkane Carbon Number or Equivalent Alkane Carbon Number. The sensory attributes may be defined as described in “Sensory Evaluation Techniques”, Morten C. Meilgaard, Gail Vance Civille, B. Thomas Carr, 5th edition, CRC Press, Taylor & Francis Group, Boca Raton, ISBN: 978-1-48221691-2 (e-book) 2016. Sensory attributes determined after 20 minutes after application were denoted with the prefix “20_”, wherein sensory attributes determined immediately after application are denoted without this prefix. The sensory attributes were determined as follows: For the sensory attribute “spreadability”, the distribution of the emollient was determined after several rubs such as three, five or twenty rubs. The sensory evaluation was done regarding an easy moving on skin (e.g. like Cetiol® Ultimate) or a difficult spreading (e.g. like Cegesoft® PS6). For the sensory attribute “wetness”, an amount of water which is perceived was determined. An oil can have a wet appearance if it is very thin and is a little cooling. The sensory attribute “thickness” of the film may be determined after 12 rubs. If there is a residue of a high layer or a low layer. If the panelist can feel directly the skin surface or if there is a film between fingertip and skin surface. The sensory attribute “oil” or oiliness may be determined after 15-20 rubs. There can be thin or rich oils. The amount of oil during rub-out is determined. A thin, pliable coating is felt that is slippery and provides a smooth, continuous feel. For the sensory attribute “stickiness”, tackiness of the oil on the skin was determined after absorption. The degree to which fingers adhere to residual product may be assessed. For the sensory attribute “amount of residue”, an amount of product that is felt on the skin immediately after absorption may be determined. The sensory attribute “thickness of residue” may be determined after the oil is absorbed, in particular time immediately after absorption, like in the rub-out phase. For the sensory attribute “powdery feel”, it was determined if there is a very dry, smooth and silky feel on the skin. There is a thin, slippery coating on the skin that leaves a very dry, smooth and silky feel on the skin. It reminds of talcum powder or baby powder or corn starch. The sensory attribute “silicone feel” may refer to a pliable coating that provides a continuous feel that fills in the texture of the skin and obscures fingerprint feel. Can be thick or thin. The coating is usually very persistent and hard to remove, if there is any. Reminiscent of silicone oil feel. The sensory attribute “smoothness” may be determined directly after the oil is absorbed and may refer to a degree to which the skin feels smooth while gliding over the arm is evaluated. The sensory attribute “dryness”, also denoted as “wax-dry”, may be determined after the oil is absorbed. A thin, stiff coated feel that is draggy, but not slippery. It provides a smooth feel on the skin with an occlusive barrier. It can be also dry with a hint of drag, reminiscent of a wax candle. The sensory attribute “grease”, also denoted cushiony, of the skin may be determined after 15-20 rubs. If there is a nourishing feel or a soft feel. The feeling is a reminiscent of a pillow. The sensory attribute “slipperiness”, also referred to as “gliding”, may refer to a measure of an ease of moving fingers across the skin. The sensory attribute “rubs to absorbency” may refer to a number of rubs until the panelist feels that the oil is absorbed. The sensory attribute “gloss” may be determined after the oil is absorbed. The panelist may assess if the skin is shiny, e.g. like satin.

The sensory attributes were measured by trained panelists, wherein test conditions with respect to temperature, humidity and the like are selected according to “Basic Principles of Sensory Evaluation”, ASTM Special Technical Publication 433 (1969) and/or DIN 10950:2020-09, “Sensorische Prüfung-Allgemeine Grundlagen incl. Anforderungen an die Räumlichkeiten”.

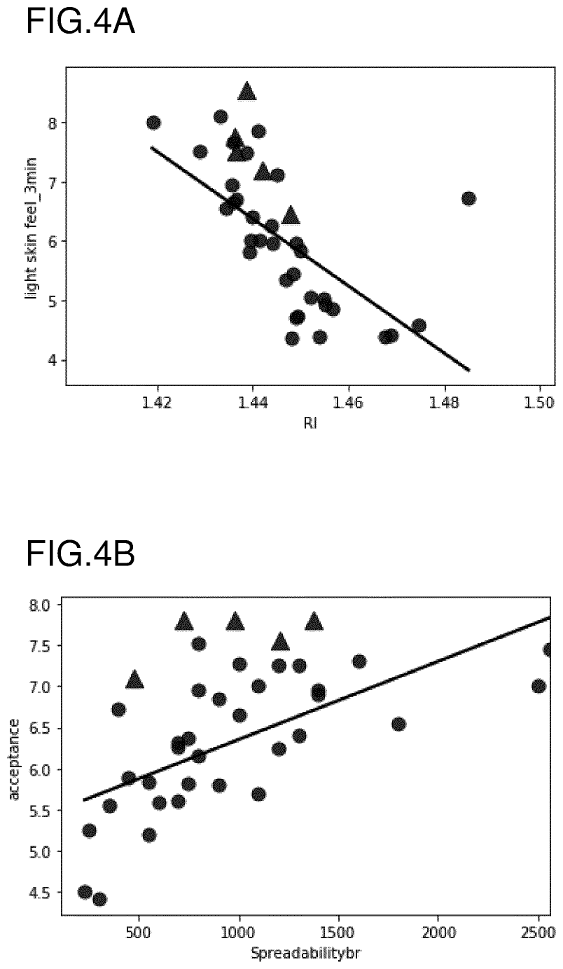

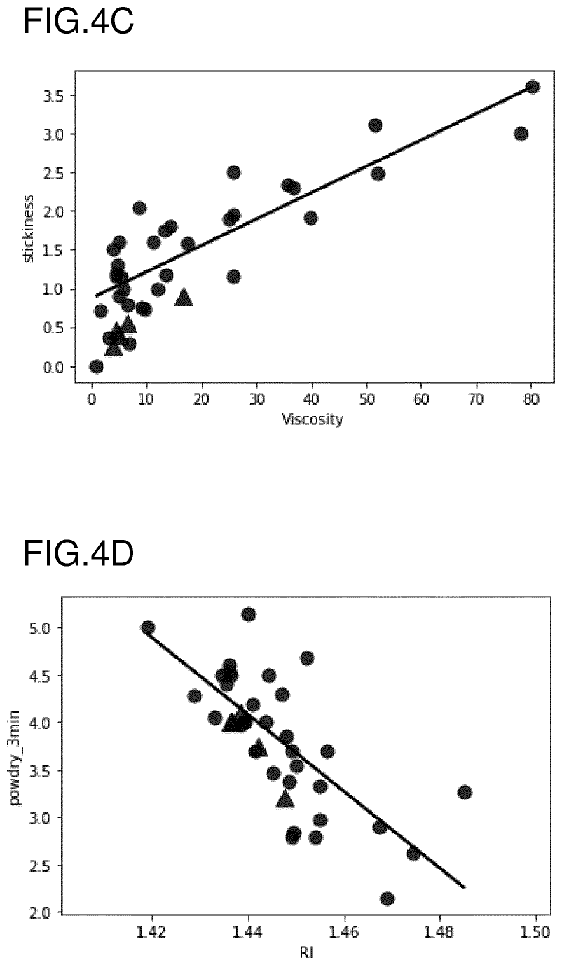

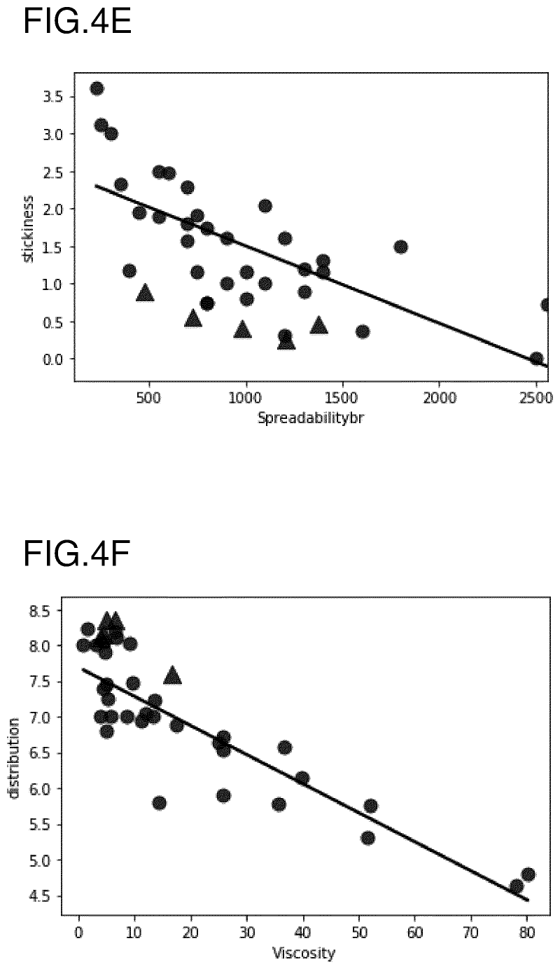

FIGS. 4A to 4F show further exemplary correlation plots. As for FIGS. 3, 20 trained panelists were used for determining the sensory attributes who applied the emollients on their forearms and provided a values of the individual attributes on a monadic scale. FIG. 4A shows “Light skin feel 3 min” (high or low light feeling (in contrary to rich feeling) of the skin after 3 min) vs the refractive index “RI”. FIG. 4B shows “Acceptance” (liking or disliking of the product) vs spreadability “spreadabilitybr”. FIG. 4C shows stickiness (low or high stickiness (or tackiness) of the fingers on the skin) vs viscosity. FIG. 4D shows Powdery 3 min “powdry_3min” (high or low powdery feeling of the skin after 3 min) vs refractive index “RI”. FIG. 4E shows stickiness (low or high stickiness (or tackiness) of the fingers on the skin) vs spreadability “spreadabilitybr”. FIG. 4F shows distribution (easy or difficult to be distributed (or spread) during rub-in phase on the skin) vs viscosity. In FIGS. 4A to 4F circles denote an oil; triangles denote a mixture. The following mixtures were used Cetiol® OE Cetiol® LC 1:3, Myritol® 318 Cetiol® OE 1:3, Cetiol® OE Cetiol® LC 1:1, Cetiol® LC Myritol® 318 1:3, Cetiol® OE Cetiol® LC 3:1. These Figures show that for oils and for mixture a correlation can be observed and used for prediction.

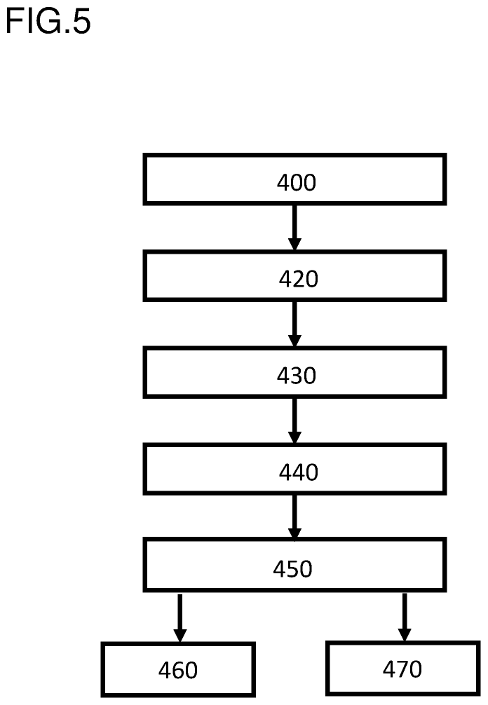

FIG. 5 shows an example of a flowchart for monitoring and/or controlling quality of a personal care product comprising a mixture of oils. In a first step 400 physico-chemical properties of the mixture of oils are provided. The physico chemical properties of the mixture of oils may be derived from composition parameters, and the physico chemical properties of each of the oils in the mixture or from measurements of physico-chemical properties of the mixture of oils. In a second step 420, sensory attributes are determined based on the physico-chemical properties of the mixture of oils, e. g. as described with respect to FIG. 2. In a third step 430 target sensory attributes are provided, In a fourth step 440, the determined sensory attributes are compared to the target sensory attributes.

In a fifth step 450 the determined sensory attributes are used for validation and the target sensory attributes. Such validation may be performed by comparing values or value ranges. If the values lie within an acceptable range or value, such as a 1- or 2-standard deviation(s) interval, the personal care product as measured may be valid in the sense that it fulfils the performance criterium or criteria. If the values do not lie within an acceptable range, such as a 1- or 2-standard deviation(s) interval, the fragrance product as measured may be invalid in the sense that it does not fulfil the performance criterium or criteria.

If the personal care product is valid, e.g. a control signal for a production process may be triggered in step 460. Such control signal may be associated with the composition of the personal care product. It may control dosing equipment for dosing of different components of the personal care product in the production process.

If the personal care product is invalid, e.g. a warning signal for the operator of the production process may be triggered in step 470. Such warning signal may signify the invalidity of the personal care product. The invalidity may trigger a stop signal for the production process.

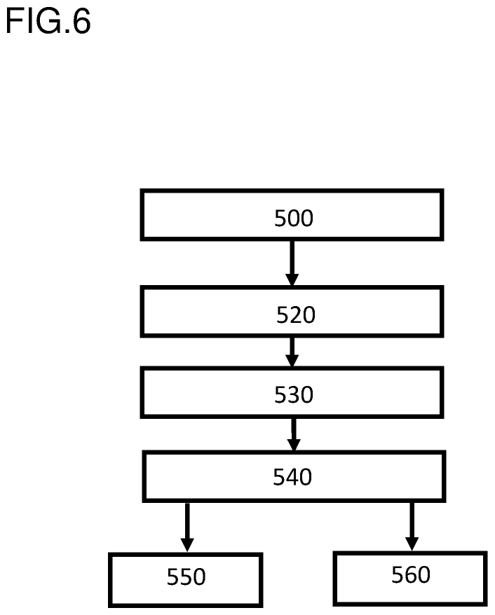

FIG. 6 shows an example of the flowchart for validating the production of a personal care product comprising a mixture of oils with at least one new ingredient based on the physicochemical properties personal care product. In a first step 500 existing sensory attributes for a personal care product that has been produced are provided, e. g generated according to the method described in FIG. 2. In a second step 520, physico-chemical properties for a new personal care product associated with at least one new ingredient are provided, these may be measured or determined from the physico-chemical properties of each oil in the mixture of oils. In a third step 530, new sensory attributes associated with the physico-chemical properties are provided. The new sensory attributes may be generated according to the method of FIG. 2. In a fourth step 540 the new and the existing sensory attributes are compared to validate the new ingredient(s). If the comparison lies within an acceptable range, the new ingredient is valid. If the comparison does not lie within an acceptable range, the new ingredient is not valid. If the new ingredient(s) is valid, e.g. a control signal for a production process based on the new ingredient(s) may be triggered in step 550. Such control signal may by be associated with the composition of the personal care product including the new ingredient. It may control dosing equipment configured to dose different components of the personal care product in the production process.

If the personal care product is invalid, e.g. a warning signal for the operator of the production process may be triggered in step 560. Such warning signal may signify the invalidity of the new ingredient(s). This may trigger a stop signal for the production process. The new ingredient may be a new oil.

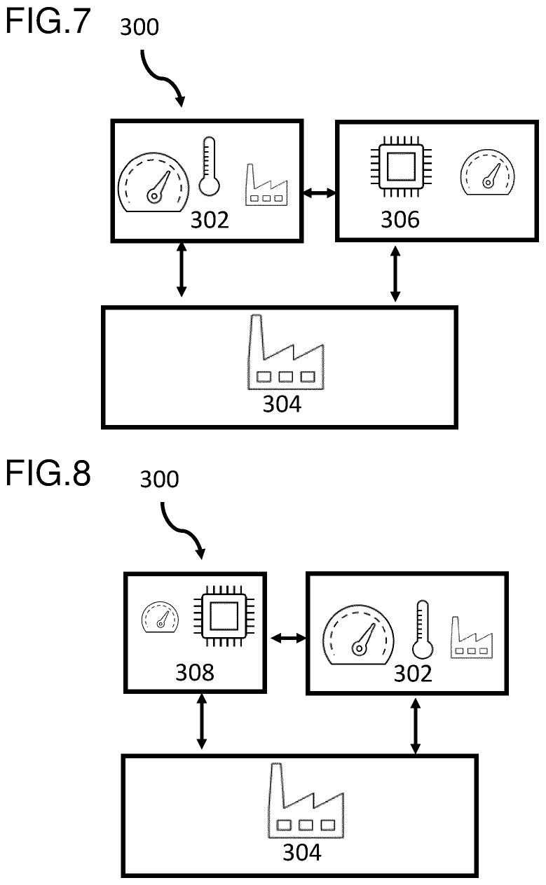

FIG. 7 shows an example of a production line 300 for producing a personal care product comprising a mixture of oils with a monitoring apparatus 306.

The production line 300 may comprise dosing equipment 302 configured to dose different ingredients of the personal care product comprising a mixture of oils, in the production process. The ingredients may comprise one or more oils. The production line may comprise a conveyor system 304 to convey e.g. bottles, plastic packaging or other suitable packaging to be filled with the personal care product. The production line may comprise a monitoring apparatus 306 configured to monitor quality of the personal care product in a production process of the personal care product based on sensory attributes. The sensory attributes may be the most relevant quality criteria for a personal care product.

The monitoring apparatus 306 and/or the dosing equipment apparatus 302 may be configured to obtaining physico-chemical properties of the mixture of oils. Physico-chemical properties of the mixture of oils may be determined based on the physico-chemical properties of each of the oils in the mixture of oils as based on composition parameters, wherein composition parameters may be derived based on feed rates of oils or may be obtained by measuring physico-chemical properties of the mixture of oils. The sensory attributes may be represented as sensory attribute data. Levels for specific sensory attributes or combinations of sensory attributes may be considered quality criteria, e.g. a quality criteria may be the skin feel. The monitoring apparatus 306 may be configured to provide the composition data, in other words the composition parameters, to the dosing equipment 302 and vice versa. The dosing equipment 302 may be configured to control the dosing based on the provided composition data.

The monitoring apparatus 306 may be configured to measure one or more physico-chemical properties. The monitoring apparatus 306 may be configured to compare the determined sensory attribute(s), to target sensory attribute(s). If the comparison lies within an acceptable range or value, the produced personal care product fulfills quality criteria. If the comparison does not lie within an acceptable range or value, the produced personal care product does not fulfill quality criteria. In the latter case, the monitoring apparatus 306 may be configured to notify an operator or to provide adjusted composition parameters to the dosing equipment 302. Adjusted composition parameters may be determined according to embodiments 9-11.

FIG. 8 shows another example of a production line 300 for producing the mixture of oils product with a validation apparatus 308.

The production line 300 may comprise dosing equipment 302 configured for dosing different ingredients of the personal care product comprising a mixture of oils, in the production process. The ingredients may comprise one or more oils. The production line may comprise a conveyor system 304 to convey e.g. bottles, plastic packaging or other suitable packaging to be filled with the personal care product. The production line 300 may comprise a monitoring apparatus 306 configured for monitoring quality of the personal care product in a production process of the personal care product based on sensory attributes. The sensory attributes may be the most relevant quality criteria for a personal care product. The production line 300 may comprise a validation apparatus 308 configured for validating the production of the personal care product based on the sensory attributes.

The validation apparatus 308 may be configured for receiving one or more data associated with new ingredient(s). The validation apparatus 308 may be configured for generating a new sensory attribute based on the provided data related to the new ingredient(s). The validation apparatus 308 may be configured to receive existing sensory attributes. The validation apparatus 308 may be configured to compare the existing and the new sensory attribute. The validation apparatus 308 may be configured for validating the new ingredient(s) for production of the mixture of the personal care product based on such comparison. The validation apparatus 308 may be configured to provide the composition data including the new ingredient(s) to the dosing equipment 302 and vice versa.

Combinations and modifications of the embodiments shown in FIGS. 7 and 8 are similarly possible. Both methods exemplify the strength of the methods described herein. The generation of the sensory attributes of a personal care product allow for objective assessment of the product in production, since the sensory attributes can be derived from objective physico-chemical properties of the mixture of oils product. This allows for simplified and more reliable production through monitoring production of the personal care product or through validating new ingredients(s) to be used for producing the personal care product.

### LIST OF REFERENCE NUMBERS

110 system

112 communication interface

114 processing device

116 input device

118 output device

120 memory

122 testing unit

124 mixing module

126 obtaining

128 determining

130 providing

300 production line

302 dosing equipment

304 conveyor system

306 monitoring apparatus

308 validation apparatus

400 first step

420 second step

430 third step

440 fourth step

450 fifth step

460 step

470 step

500 first step

520 second step

530 third step

540 fourth step

550 step

560 step

1. System A system (110) for predicting sensory attributes or physico-chemical properties of an oil or a mixture of oils for personal care, wherein the system (110) comprises at least one communication interface (112) for providing data and at least one processing device (114), wherein the processing device (114) is configured for:
obtaining via the communication interface (112) of at least one physico-chemical property of the oil or the mixture of oils or of at least one sensory attribute and of at least one model relating one or more physico-chemical properties to one or more sensory attributes;
determining at least one sensory attribute of the oil or the mixture of oils based on the obtained physico-chemical properties and the model or determining at least one physico-chemical property of the oil or the mixture of oils based on the obtained sensory attributes and the model;
providing via the communication interface (112) the determined sensory attribute or the determined physico-chemical property.

2. The system (110) according to claim 1, wherein the mixture of oils comprises at least two different oils, at least three different oils, or at least four different oils.

3. The system (110) according to claim 1, wherein the determined sensory attribute comprises sensory attributes for each of the oils.

4. The system (110) according to claim 1, wherein the determined physico-chemical property comprises physicochemical properties for each of the oils.

5. The system (110) according to claim 1, wherein the processing device (114) is configured for obtaining via the communication interface (112) at least one unique identifier of the oil and/or mixture of oils, wherein the processing device (114) is configured for retrieving the physico-chemical properties or the sensory attributes for the oil and/or mixture of oils defined by the obtained unique identifier.

6. The system (110) according to claim 5, wherein the processing device (114) is configured for determining the at least one sensory attribute for the oil and/or mixture of oils defined by the obtained chemical formulation and/or the unique identifier based on the retrieved physico-chemical properties and the model, wherein the processing device (114) is configured for providing via the communication interface the at least one sensory attribute for the oil and/or mixture of oils defined by the obtained chemical formulation and/or the unique identifier.

7. The system (110) according to claim 1, wherein the processing device (114) is configured for obtaining via the communication interface (112) at least one target sensory attribute, wherein the processing device (114) is configured for determining at least one physico-chemical property based on the obtained sensory attributes and the model, wherein the processing device (114) is configured for determining a target oil or a target ratio of a mixture of oils having the obtained target sensory attribute based on the determined physico-chemical property.

8. The system (110) according to claim 1, wherein the processing device (114) is configured for obtaining via the communication interface (112) at least one target physico-chemical property, wherein the processing device (114) is configured for determining at least one sensory attribute based on the obtained physico-chemical property and the model, wherein the processing device (114) is configured for determining a target oil or a target ratio of a mixture of oils having the obtained target physico-chemical property based on the determined sensory attribute.

9. The system (110) according to claim 1, wherein the processing device (114) is configured for determining a composition of a cosmetic product comprising the oil and/or mixture of oils based on the provided physico-chemical property or the provided sensory attribute.

10. The system (110) according to claim 9, wherein the cosmetic product is a personalized cosmetic product.

11. The system (110) according to claim 1, wherein the communication interface (112) comprises at least one web interface configured for providing at least one input box for inputting the at least one physicochemical property of the oil or the mixture of oils or of the at least one sensory attribute.

12. The system (110) according to claim 1, wherein processing device (114) is configured for providing via the communication interface (112) a formulation of the mixture relating to the determined sensory attribute or the determined physico-chemical property.

13. A computer implemented method for predicting sensory attributes or physico-chemical properties of an oil or a mixture of oils for personal care by using at least one system (110) according to claim 1, wherein the method comprises:
a) obtaining via the communication interface (112) of at least one physico-chemical property of the oil or the mixture of oils or of at least one sensory attribute and of at least one model relating one or more physico-chemical properties to one or more sensory attributes by using the processing device (114);
b) determining at least one sensory attribute of the oil or the mixture of oils based on the obtained physico-chemical properties and the model or determining at least one physico-chemical property of the oil or the mixture of oils based on the obtained sensory attributes and the model by using the processing device (114);
c) providing via the communication interface (112) the determined sensory attribute or the determined physico-chemical property by using the processing device (114).

14. A computer program including computer-executable instructions for performing the method according to claim 13 when the program is executed on a computer or computer network.

15. A computer program product having program code means, in order to perform the method according to claim 13 when the program is executed on a computer or computer network.

16. A method for producing a personal care product comprising using a determined sensory attribute or a determined physico-chemical property determined according to claim 13.
